(19) United States
(12) Patent Application Publication
(10) Pub. No.: US 2009/0136538 A1
(43) Pub. Date: May 28, 2009

(54) Stable vaccine formulation

(76) Inventor: Jan Jezek, Wellingborough (GB)

Correspondence Address: ELMORE PATENT LAW GROUP, PC, 515 Groton Road, Unit 1R, Westford, MA, 01886, US

(21) Appl. No.: 12/323,708

(22) Filed: Nov. 26, 2008

Related U.S. Application Data
(63) Continuation-in-part of application No. 12/275,690, filed on Nov. 21, 2008.
Application No. 12/275,690 is a continuation of application No. PCT/GB2007/001898, filed on May 22, 2007.

(30) Foreign Application Priority Data
May 22, 2006 (GB) 0610140.6
Jul. 3, 2006 (GB) PCT/GB2006/002470

(51) Int. Cl. A61K 39/385; C07K 1/04

(52) U.S. Cl. 424/193.1; 424/196.11; 530/403

(57) ABSTRACT

An aqueous vaccine composition comprises a protein adsorbed on a solid and one or more stabilising agents, further characterized in that
(i) the system is optionally substantially free of a conventional buffer, i.e a compound with pKₐ within 1 unit of the pH of the composition at the intended temperature range of storage of the composition;
(ii) the pH of the composition is set to a value at which the composition has maximum measurable stability with respect to pH; and
(iii) the one or more additives are capable of exchanging protons with the said protein and have pKₐ values at least 1 unit more or less than the pH of the composition at the intended temperature range of storage of the composition.

### RELATED APPLICATION

This application is a continuation-in-part of U.S. Ser. No. 12/275,690 filed on Nov. 21, 2008 which is a continuation of PCT/GB2007/001898 filed on May 22, 2007, which claims priority under 35 U.S.C. § 119 or 365 to United Kingdom Application No. 0610140.6, filed on May 22, 2006 and United Kingdom Application No. PCT/GB2006/002470, filed on Jul. 3, 2006. The entire teachings of the above applications are incorporated herein by reference.

### FIELD OF THE INVENTION

This invention relates to stable vaccine formulations comprising proteins.

### BACKGROUND OF THE INVENTION

The loss of a protein's native tertiary structure is generally associated with the loss of its biological activity. It is therefore crucial to ensure that an active protein (e.g. vaccine, therapeutic protein, diagnostic protein etc.) is stored under conditions where the native tertiary structure is maintained.

Storage of proteins for any length of time poses stability problems. The fluctuations of the tertiary structure are proportional to the temperature. Proteins are therefore generally more stable at lower temperatures. Typically, proteins have to be stored freeze-dried (lyophilised) or frozen (around −20° C.) to preserve their biological activity. If stored freeze-dried or frozen, the protein has to be reconstituted before its use. For short-term storage of proteins, refrigeration at 4° C. may be sufficient.

Proteins are macromolecules consisting of sequences of 20 different naturally occurring amino acids. Seven of these amino acids (aspartic acid, glutamic acid, histidine, cysteine, tyrosine, lysine and arginine) contain a side-chain capable of engaging in acid-base equilibria. This means that they can either accept or donate a proton depending on pH and other species present in the solution. Serine and threonine are also capable of exchanging protons with the surrounding molecules. However, the pKₐ values of these amino acids are extremely high (>13.9), so their side-chains are practically always in almost totally protonated form.

The immunogenic activity of protein vaccines depends (to a large extent) on the structural integrity of the key protein antigens, especially in relation to conformational epitopes (where antibodies are required to bind disparate regions of the polypeptide chain brought together by native folding). Irreversible conformational changes and irreversible aggregation lead to inactivation of vaccines. The same considerations apply to proteins adsorbed onto particles, such as alumina particles, or other (non-particulate) surfaces when substantial regions of each protein molecule are still in full interaction with solvent water.

Existing vaccines currently used in immunization programs are heat sensitive and require a cold chain for transportation and storage. Maintaining the cold chain at a specific temperature range, normally 2° C. to 8° C., is both costly and challenging for resource-constrained countries. The temperature sensitivity of vaccines creates complex logistics for immunization programs trying to reach rural populations, and in certain cases limits immunization coverage. The unreliability and breakage of the cold chain in the periphery may result in vaccine damage or wastage, not only in developing countries, but also in places with established cold chains where spikes in temperature may occur due to various interruptions. These problems can be partially or wholly alleviated if vaccines are thermally stable.

Hepatitis B vaccine is one of the most heat-stable children's vaccines maintaining stability for up to 4 years at temperatures between 2° C. and 8° C. A 50 percent loss of potency of the vaccine has been reported to be observed after 9 months at 20° C. to 26° C., after 1 month at 36° C. to 40° C., and after 3 days at 45° C. By taking advantage of the relative thermostability of hepatitis B vaccine, several countries, including Vietnam, Indonesia, and China, have been experimenting with out-of-cold chain use for immunizing infants with the birth dose. Vaccines are kept by midwives or clinics without refrigeration for up to 1 month and administered to infants shortly after birth. Results have shown that this out-of-cold chain use has improved the coverage of the birth cohort without compromising the effectiveness of the vaccine. Improvements to the heat stability of hepatitis B vaccine would further ease immunization logistics and allow even wider and safer immunization coverage, especially in tropical climates.

Commercially available Hepatitis B vaccine is a liquid suspension consisting of purified recombinant hepatitis B surface antigen (HBsAg) adsorbed onto aluminum hydroxide adjuvant. HBsAg is a recombinant non-glycosylated lipoprotein complex, which is the primary viral envelope protein responsible for immunogenicity and immunity from hepatitis B virus infection. The recombinant HBsAg polypeptide has a molecular weight of 24 kDa, and self-assembles readily in solution to form liposome-like 22 nm particles presenting antigenic HBsAg epitopes on their surface (R. A. Rader. Biopharmaceutical Products in the US and European Markets. Bioplan Associates Inc., Rockville, Md., 6th edition (2007)). HBsAg has an isoelectric point of about 4.5, while aluminum hydroxide adjuvant has a point of zero charge of 11.4. Consequently, electrostatic adsorption is conceivable between the adjuvant and the antigen at a wide range of pH at which the HBsAg is negatively charged and aluminum hydroxide adjuvant is positively charged. The adsorption of HBsAg by aluminum hydroxide adjuvant was shown to exhibit a high affinity adsorption isotherm (S. Iyer, R. S. R Robinett, H. HogenEsch, and S. L. Hem. Mechanism of adsorption of hepatitis B surface antigen by aluminum hydroxide adjuvant. Vaccine. 22: 1475-1479 (2004)). However, in spite of the favourable electrostatic properties, the adsorption of HBsAg by aluminum hydroxide adjuvant was shown to be predominantly due to ligand exchange between the phospholipids of HBsAg and surface hydroxyls of aluminum hydroxide adjuvant. Electrostatic interactions and hydrophobic interactions have been shown to play a considerably less important role. The surface characteristics of the adjuvant particle can be modified considerably in the presence of phosphate anions due to a strong affinity of phosphate to the aluminum cation and consequent ligand exchange between the phosphate anion and hydroxide anion (P. M. Callahan, A. L. Shorter, and S. L. Hem. The importance of surface charge in the optimization of antigen-adjuvant interactions. Pharm Res. 172: 121-130 (1991)). Such modification lowers the point of zero charge of the adjuvant causing the particle surface to be less positively charged at a given pH. Consequently, the adsorption of anions from the bulk solution to form the stem layer surrounding the particle is suppressed. It has been suggested previously (A. Wittayanukulluk, D. Jiang, F. E. Regnier, and S. L. Hem. Effect of microenvironment pH of aluminum hydroxide adjuvant on the chemical stability of adsorbed antigen. Vaccine. 22: 1172-1176 (2004)) that this effect results in changes in pH microenvironment at the surface of adjuvant particle due to lower adsorption of the hydroxide anion.

A need exists to produce stable vaccines that are not reliant on maintaining a cold chain.

### SUMMARY OF THE INVENTION

According to the invention, a vaccine composition comprises a protein and one or more stabilising agents. In one embodiment, the vaccine comprises a protein adsorbed on a solid, such as an adjuvant. The stabilised protein may be in a microbiologically sterile form, and is conveniently contained or stored in a sealed, sterile container such as a vial, syringe or capsule.

In one embodiment of the present invention, an aqueous system comprises a protein and one or more additives, characterised in that
(i) the system is substantially free of a conventional buffer, i.e a compound with pKₐ within 1 unit of the pH of the composition at the intended temperature range of storage of the composition;
(ii) the pH of the composition is set to a value at which the composition has maximum measurable stability with respect to pH;
(iii) the one or more additives are capable of exchanging protons with the said protein and have pKₐ values at least 1 unit more or less than the pH of the composition at the intended temperature range of storage of the composition.

By keeping a protein at a suitable pH, at or near a value at which the measurable stability is maximal, in the absence of a conventional buffer, the storage stability of the protein can be increased substantially. Storage stability can generally be enhanced further, possibly substantially, by use of additives with pKₐ values having 1 to 5 units away from the pH of the composition at the intended temperature range of storage of the composition. The presence of these additives also improves the pH stability of the formulation and is generally preferred.

In accordance with the present invention the protein composition does not comprise a conventional buffer in a meaningful amount and, thereby, it is substantially free of the conventional buffer. In other words, the protein composition contains less than a meaningful amount of the conventional buffer. Conventional buffers are typically applied in protein compositions at concentrations 2-200 mM, more typically at 5-50 mM and most typically at about 20 mM concentration. The term “conventional buffer” is therefore defined herein as any chemical species with pKₐ less than one unit but preferably less than 0.5 unit away from pH of the composition as measured at the intended temperature range of storage of the composition which possesses a buffering capacity for the protein. The term “less than a meaningful amount” means that the conventional buffer is generally present in the composition at concentration less than 5 mM, but preferably less than 2 mM, such as an amount that does not provide substantial buffering capacity. The composition can be said to be “free” of such a buffer if the composition if no additional buffer has been added.

Preferably, the composition contains one or more additives capable of engaging in acid-base equilibria either with pKₐ values at least 1 unit below the pH of the composition and/or with pKₐ values at least 1 unit above the pH of the composition. As used herein, one or more units above or below the pH of the composition are also referred to herein as 1 or more units “away” from the pH of the composition. Such additives can protect the composition from significant shifts of pH either toward acidic values (if pKₐ is lower than pH of the composition) or toward alkaline values (if pKₐ is higher than pH of the composition). In one embodiment, additives include, but are not limited to, “displacement buffers” in accordance with the invention.

Most preferably, the composition contains one or more additives capable of engaging in acid-base equilibria both with pKₐ values at least one unit below and with pKₐ values at least one unit above the pH of the composition. Such additives can protect the composition from significant shifts of pH toward both acidic and alkaline values. Such additives are suitably present in an amount such that the molarity of each additive is at least 1 mM and/or less than 1 M, preferably 2 mM to 200 mM, most preferably 5 mM to 100 mM. In one embodiment, one or more additives are preferably present at a concentration of 1 mM to about 1M; more preferably at a concentration of from about 2 mM to about 200 mM, and even more preferably at a concentration from about 5 mM to about 100 mM. See also, WO2007/003936, which is incorporated herein by reference.

In the case of HBsAg, the pH of the formulation is preferably between about 4 and about 6, more preferably between about 4.7 and about 5.7, such as about 5.2. Preferred stabilizing additives for HBsAg can be histidine and lactate. In a preferred embodiment, the vaccine formulation contains at least about 20 mM, preferably at least about 30 mM, more preferably at least about 40 mM of each histidine or lactate.

Additives are suitably present in an amount such that the total concentration of additives is in the range 1 mM to 1 M, preferably 1 mM to 200 mM, most preferably 5 mM to 100 mM. In certain practical applications, especially in medical applications, it will often be desirable to use as low concentrations of additives as possible.

Without being bound by theory, the presence of phosphate anion in a typical vaccine formulation, as discussed above, can have a profound effect on the interactions between the antigen adsorbed on an aluminum hydroxide adjuvant particle and its surrounding environment. It is therefore believed that the pH-related interactions affecting the stability of an antigen adsorbed on aluminum hydroxide adjuvant can be controlled by adjusting the level of phosphate in the formulation. In a preferred embodiment, the vaccine formulation contains at least about 20 mM, preferably at least about 30 mM, more preferably at least about 40 mM of phosphate.

Based on the nature of a vaccine, such as the hepatitis B vaccine and the mechanism of stability breakdown, it appears possible to further improve the stability of the vaccine through changes in formulation. This can be achieved both by modifying the interaction between the recombinant antigen and the adjuvant by adjusting the level of phosphate, and by further formulation optimization, including optimization of pH and addition of other components, ensuring native conformation of the antigen is maintained.

The invention thus enables improvements to be made in the storage stability of vaccines, such as antigenic proteins absorbed on adjuvants, in aqueous environment such that proteins can be stored for extended periods of time at ambient temperatures without significant loss of activity, thus avoiding the need for freezing or refrigeration.

### BRIEF DESCRIPTION OF THE DRAWINGS

FIG. 1 shows that the stability of adsorbed HBsAg was shown to be strongly dependent on the concentration of phosphate in the formulation (FIG. 1). Less than 20 percent of the original antigenic activity could be recovered after 2 weeks and less than 8 percent after 9 weeks of incubation at 55° C. in the absence of phosphate across the pH range studied. In contrast, more than 50 percent of the original antigenic activity was recovered after incubation of the vaccine in 20 mM phosphate at 55° C. for 2 weeks and more than 20 percent after incubation for 9 weeks.

FIG. 2 shows that the presence of succinate anion had a small beneficial effect on the HBsAg stability resulting in greater than 60 percent recovery of the antigenic activity following incubation at 55° C. for 9 weeks at pH 4.6 and 5.2. In contrast, the presence of lactate anion and particularly the presence of histidine resulted in considerably improved stability of HBsAg.

FIG. 3 shows that the antigenic activity of the Shanvac-B vaccine dropped to approximately 60 percent after 1 month at 37° C. and about 30 percent after 6 months at 37° C. Similarly, the activity following incubation at 45° C. dropped to about 40 percent after 1 month and less than 10 percent after 6 months.

FIG. 4 shows the ability of the displacement buffers to maintain pH away from their respective pKₐ values increases with their concentration.

FIG. 5 shows a titration curve of a composition titrated with either a base (OH⁻) or an acid (H₃O⁺).

### DESCRIPTION OF PREFERRED EMBODIMENTS

Certain preferred features of the invention are defined in the subclaims. It will be appreciated that this invention relates to the stability of proteins, particularly the stability of proteins in an aqueous environment, e.g. in aqueous solution, in aqueous gel form and in solid state (or other non-liquid state) where free or bound water is present, and concerns the storage stability of proteins, i.e. stability with time, including stability at ambient temperatures (about 20° C.) and above. The term “protein” is used herein to encompass molecules or molecular complexes consisting of a single polypeptide, molecules or molecular complexes comprising two or more polypeptides and molecules or molecular complexes comprising one or more polypeptides together with one or more non-polypeptide moieties such as prosthetic groups, cofactors etc. The invention is particularly advantageous to proteins absorbed on adjuvants, such as aluminum hydroxide, or other solid which creates a pH microenvironment substantially different from (e.g., >0.5) the pH of the bulk liquid media. Preferred proteins are those used to make vaccines, e.g., antigens.

The invention is applicable to any system in which the retention of structural characteristics of a protein, in particular the secondary, tertiary and quaternary structure, and the retention of functional characteristics of the protein, in particular the antigen or receptor binding, are of importance.

The application of the invention reduces significantly the probability of irreversible conformational change and irreversible aggregation of a protein and consequent loss of protein activity.

The invention is applicable to stabilisation of a protein throughout its product life including isolation or expression, purification, transport and storage.

In terms of molecular size, the invention is applicable to polypeptides with a relative molecular weight of at least 2000 where at least basic motifs of secondary or tertiary structure are likely to be formed. There is no upper limit of the relative molecular weight that would limit application of the present invention.

In terms of secondary structure, the invention is applicable to proteins containing any proportion of alpha helix, beta sheet and random coil.

In terms of tertiary structure, the invention is applicable both to globular proteins and to fibrillar proteins. The invention is applicable to proteins whose tertiary structure is maintained solely by means of non-covalent interactions as well as proteins whose tertiary structure is maintained by combination of non-covalent interactions and one or more disulphide bridges.

In terms of quaternary structure, the invention is applicable to monomeric proteins as well as proteins consisting of two, three, four or more subunits. The invention is also applicable to protein conjugates.

In terms of non-protein structural components, the invention is applicable to proteins that do not contain any non-peptide components as well as glycoproteins, lipoproteins, nucleoproteins, metalloproteins and other protein containing complexes where protein represents at least 10% of the total mass. It is applicable to proteins that do not require a cofactor for their function as well as to proteins that require a coenzyme, prosthetic group or an activator for their function.

The invention is applicable to proteins attached to solid substrates such as vaccine adjuvant by means of hydrophobic, ionic or ligand exchange interactions. The invention is also applicable to proteins dissolved in aqueous gel form and proteins in solid state where water has been removed partially or fully from an aqueous solution by drying or by freeze-drying where free or bound water is still present.

The protein may be native or recombinant, glycosylated or non-glycosylated, autolytic or non-autolytic. The invention is particularly applicable to recombinant protein vaccines as well as attenuated viruses or whole cell vaccines, providing the key antigens consist of polypeptide chains. Phosphoproteins or glycoproteins are preferred. Examples of such vaccines include:

Hepatitis B vaccine

Malaria vaccine

Human papilloma vaccine

MeningitisA vaccine

Meningitis C vaccine

Pertussis vaccine

Polio vaccines

The protein used in the invention can be maintained substantially in its native state. For the purposes of this specification, the term “native protein” is used to describe a protein having retained tertiary structure, and distinction from proteins that have undergone a degree of unfolding or denaturation. A native protein may incorporate some chemical modification, e.g. deamidation, rather than physical modification.

The protein can be adsorbed on a solid surface such as alumina. In some cases, it is beneficial to pre-incubate the solid surface (such as alumina particles) in the stabilising formulation prior to adsorption of the protein onto the surface. This can result in greater stability of the protein.

For the particular case of immunogenic proteins used in association with an adsorbent/adjuvant such as alumina, phosphate is a preferred stabilising agent. The use of phosphate is relatively common in pharmaceutical formulations as a buffer at pH around 7. However, its application in the present invention is by no means restricted to compositions around pH 7. For example, the optimal formulation of Hepatitis B vaccine comprises phosphate anion at pH around 5.2 where its buffering capacity is negligible. The beneficial effect of phosphate can be attained fully only if a certain concentration is achieved. Such concentration is often higher than that used typically for buffering purposes, such as >20 mM, >30 mM or >40 mM.

The present invention is based in part on the discovery that buffers having a pKa at or near the pH of the solution are undesirable, when considering the protein's stability with respect to pH. Rather, the key to the present invention is choice of the appropriate pH while minimizing the protein's ability to exchange ions. Various aspects of the invention are defined in the claims.

The present invention enables improvements to be made in the storage stability of proteins by selecting an appropriate pH of the composition without the use of a conventional buffer. The term “conventional buffer” is used herein to encompass any compound possessing a buffering capacity when present in the composition with pKₐ less than one unit away from the pH, but preferably less then 0.5 unit, most preferably with pKₐ=pH. Both the pKₐ and the pH values used in this definition are those measured at the temperature range of the intended storage of the protein composition.

Conventional buffers are typically present in protein compositions at concentrations 2-200 mM, more typically at 5-50 mM and most typically at about 20 mM concentration. Such concentrations of conventional buffers can ensure reasonable stability of pH and can therefore be referred to as meaningful concentrations with respect to their buffering action. Consequently, apart from the above specification in terms of its pKₐ, the term “conventional buffer” additionally comprises a “meaningful concentration” aspect characterised in that the said conventional buffer is present at a concentration that is meaningful with respect to a reasonable buffering action. In other words, a meaningful concentration of a conventional buffer is that concentration wherein the conventional buffer provides the predominant buffering mechanism of the system.

The present invention arose from an analysis of the effects of chemical species capable of proton exchange on stability of proteins and the subsequent development of a model that enables selection of conditions that ensure good long-term stability of proteins. The analysis revealed that the presence of acid-base species that are close to 50% protonation state is detrimental to the protein stability as determined by either functional assays or structural assays. By definition, this means that the presence of conventional buffers, especially at high concentrations, can be detrimental to the protein stability. It appears that, prior to the present invention, the adverse effect of conventional buffers on the storage stability of proteins has not been appreciated.

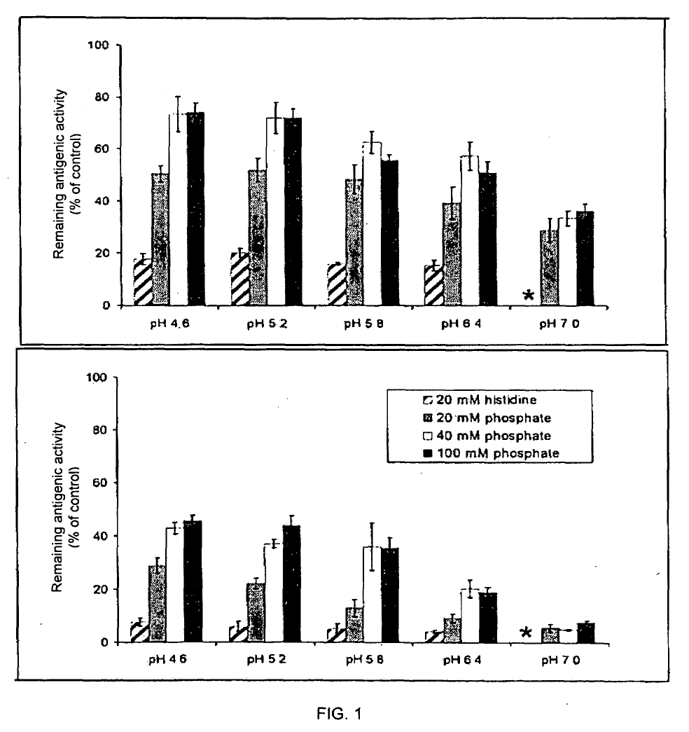

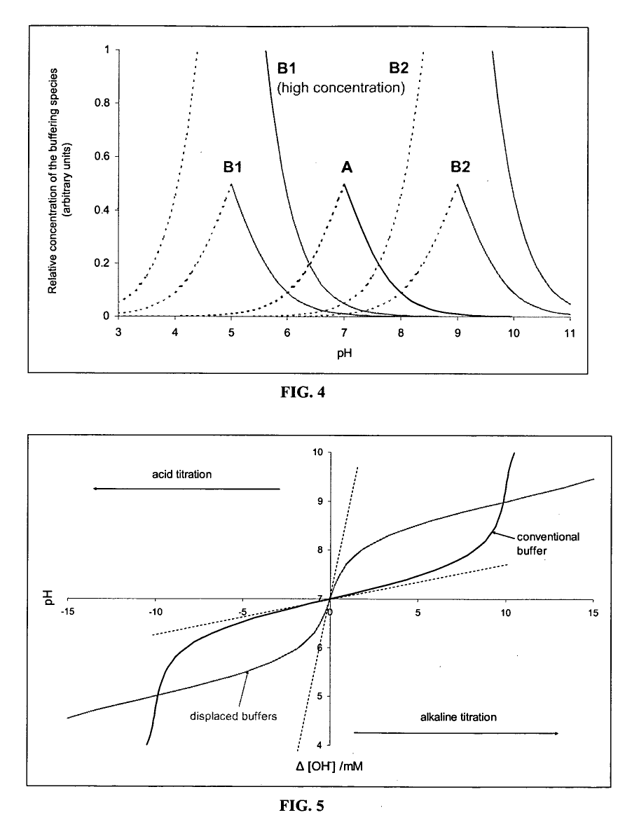

Some limited buffering capacity can be derived from the protein itself, especially in the pH range of 4.0 to 6.5 due to the side chains of aspartic acid, glutamic acid and histidine. In some cases, especially at higher protein concentrations (>20 mg mL⁻¹) this might be sufficient to maintain the required pH, especially in sterile composition in which spontaneous changes of pH are unlikely. In accordance with the invention, one or more excipients or additives can be used to maintain the required pH or minimise pH changes. This can be referred to as “displaced buffering” and is based on addition of excipients to the protein composition with pK₃ values outside the conventional buffering range, preferably excipients with pKₐ about 1 to 4 units above or below the pH of the composition. Although the “displaced buffering” cannot ensure a strong buffering capacity at the required pH comparable with the conventional buffer, it can still prevent significant fluctuations of pH away from the required value. The difference between conventional buffering and displaced buffering is shown in FIG. 1. The graph shows the relative concentrations of the buffering species of a conventional buffer (A) and displaced buffers (B1 and B2) in a hypothetical system buffered at pH 7. The dotted lines show the relative concentration of the de-protonated forms of the buffers (i.e. the form capable of preventing pH changes into acidic values); the full lines show the relative concentration of the protonated forms of the buffers (i.e. the form capable of preventing pH changes into alkaline values). The concentration of the buffering species on both the acidic and the alkaline side reflects the buffering capacity of the buffer. The conventional buffer (in this case a compound with pKₐ=7) is most effective to maintain the required pH 7. The two displacements buffers (in this case compounds with pKₐs two units above and two units below the required pH) exert minimal buffering capacity at pH 7, but their buffering capacity increases as pH moves away from 7. So, whilst these species are rather inefficient in preventing small fluctuations around the required pH they can prevent larger fluctuations away from the required pH. The ability of the displacement buffers to maintain pH away from their respective pKₐ values increases with their concentration as shown in FIG. 4.

The titration curve of the composition titrated with either a base (OH⁻) or an acid (H₃O⁺) is shown in FIG. 5. In this model example the target pH is 7, and the titration is shown of a composition comprising 20 mM of a conventional buffer (pKₐ 7) and a combination of two displaced buffers (pKₐ 5 and pKₐ 9), each at 20 mM concentration. The titration curves are theoretical ones, based on the pKₐ values and concentrations of the species present and on the assumption that no other components of the composition contribute to the buffering of the composition.

Due to a limited buffering capacity of the displaced buffers at the target pH the slope of the titration curve at the target pH (i.e. 7 in this model example) in the composition containing conventional buffer is considerably less steep compared to that containing the combination of displaced buffers. Consequently, addition of the same amount of NaOH will cause different pH change in the presence of conventional buffer compared with that in the presence of displacement buffers of the same concentration. So, in the model example shown in FIG. 5 (where the pKa of conventional buffer is precisely the same as the target pH 7) the addition of 5 mM NaOH (i.e. addition of NaOH to the composition, which results in 5 mM concentration increase of Na⁺ cations in the composition) will increase pH from 7.0 to 7.48 in the presence of conventional buffer (20 mM) and to 8.55 in the presence of displacement buffers (both at 20 mM).

In one embodiment the protein composition of the invention comprises two displacement buffers comprising at least one displacement buffer having a pKa that is at least 1 unit greater than the pH of the composition at the desired temperature and at least one displacement buffer having a pKa that is at least 1 unit less than the pH of the composition at the desired temperature. In one embodiment the protein composition of the invention comprises two displacement buffers comprising at least one displacement buffer having a pKa that is at least 1.5 units greater than the pH of the composition at the desired temperature and at least one displacement buffer having a pKa that is at least 1.5 units less than the pH of the composition at the desired temperature. In one embodiment the protein composition of the invention comprises two displacement buffers comprising at least one displacement buffer having a pKa that is at least 2 units greater than the pH of the composition at the desired temperature and at least one displacement buffer having a pKa that is at least 2 units less than the pH of the composition at the desired temperature.

In one embodiment the protein composition of the invention comprises two displacement buffers wherein each displacement buffer is from about 1 unit to about 5 units from the pH at which the protein has stability at the desired temperature. In one embodiment the protein composition of the invention comprises two displacement buffers wherein each displacement buffer is from about 1 unit to about 4 units from the pH at which the protein has stability at the desired temperature. Apart from the contribution to pH buffering, the presence of displacement buffers was shown in many cases to have a beneficial effect on the protein stability. For example, in one embodiment, protein activity of a protein in a composition in accordance with the invention retains at least 40% of its activity for at least one week, and preferably at least four weeks at a desired temperature (e.g. ambient temperature or higher). In another embodiment, protein activity of a protein in a composition in accordance with the invention retains at least 50% of its activity for at least one week at the desired temperature, and preferably at least four weeks at a desired temperature (e.g. ambient temperature or higher). In another embodiment, at least 40% and preferably at least 50% protein structural activity of a protein present in a composition according to the invention is retained for at least one week and more preferably for at least 4 weeks at the desired temperature.

The compounds that can be used as displacement buffers can be both organic and inorganic. They can be of both monomeric and polymeric nature.

Some examples of compounds that can be usefully incorporated in the protein composition as additives and that may possibly also function as displacement buffers are known and include, but are not limited to: Histidine, Maleate, Sulphite, Cyclamate, Hydrogen sulphate, Serine, Arginine, Lysine, Asparagine, Methionine, Threonine, Tyrosine, Isoleucine, Valine, Leucine, Alanine, Glycine, Tryptophan, Gentisate, Salicylate, Glyoxylate, Aspartame, Glucuronate, Aspartate, Glutamate, Tartrate, Gluconate, Lactate, Glycolic acid, Adenine, Succinate, Ascorbate, Benzoate, Phenylacetate, Gallate, Cytosine, p-Aminobenzoic acid, Sorbate, Acetate, Propionate, Alginate, Urate, 2-(N-Morpholino)ethanesulphonic acid, Bicarbonate, Bis(2-hydroxyethyl) iminotris(hydroxymethyl)methane, N-(2-Acetamido)-2, iminodiacetic acid, 2-[(2-amino-2-oxoethyl)amino]ethanesulphonic acid, piperazine, N,N′-bis(2-ethanesulphonic acid), Phosphate, N,N-Bis(2-hydroxyethyl)-2, aminoethanesulphonic acid, 3-[N,N-Bis(2-hydroxyethyl)amino]-2, hydroxypropanesulphonic acid, Triethanolamine, piperazine-N,N′-bis(2, hydroxypropanesulphonic acid), Tris(hydroxymethyl)aminomethane, N, Tris(hydroxymethyl)glycine, N-Tris(hydroxymethyl)methyl-3-aminopropanesulphonic acid, Ammonium ion, Borate, 2-(N-Cyclohexylamino)ethanesulphonic acid, 2-Amino-2-methyl-1-propanol, Palmitate, Creatine, Creatinine, and salts thereof.

The particular choice of the compound will depend on pH of the composition. So, for example, purine (pKa=9.0) is a suitable additive in a composition at pH 7.0 (where pKa−pH=2.0), but not in a composition at pH 8.8 (where pKa−pH=0.2 and purine therefore becomes the conventional buffer). It will of course be understood by one of ordinary skill in the art that aspects specific to particular proteins have to be taken into account. For instance, it is important to ensure that the additives selected do not inhibit the protein activity. Many of the suggested additives are GRAS (Generally Regarded As Safe) or approved ingredients in pharmaceutical products. These additives are particularly suitable for stabilisation of proteins in pharmaceutical compositions. Other applications where safety is not of major concern, such as proteins in diagnostic kits, may rely on compounds outside the GRAS category.

Without wishing to be bound by theory, it is believed that the beneficial effect of the present invention on the protein stability is due to the fact that at or around the 50% protonation state the acid-base species are most likely to exchange protons with surrounding acid-base species, such as some amino acids at the protein surface. Such exchanges can be detrimental to the protein for the following reasons:
Each proton exchange results in either bond formation or bond cleavage. Such processes are accompanied by energy exchanges (e.g. translational energy of the species involved) between the protein and surrounding species and by changes in charge characteristics of the part of the protein where proton is exchanged. Therefore, the continuous proton exchanges occurring when protein is in equilibrium with its aqueous environment are very likely to contribute to the fluctuations of the protein structure and consequent physical instability of the protein.
Various chemical processes affecting protein stability, such as de-amidation, involve proton exchange. Minimising the rate of these processes can therefore lead to stabilisation of the protein.

The compositions of the invention can comprise at least 0.1% (w/w), preferably at least 0.5% (w/w) of each stabilising agent. Alternatively or additionally, the compositions can comprise up to 200 mM of each stabilising agent, such as up to 100 mM of each stabilising agent. Preferred formulations having at least 20 mM, such as at least 30 mM or at least 40 mM stabilizing agent can be made.

The materials listed above are given for the purpose of illustration only. It will of course be understood by one of ordinary skill in the art that aspects specific to particular proteins have to be taken into account. For instance, it is important to ensure that the additives selected do not inhibit the protein activity. It is also important to ensure that the compounds used to improve heat stability of proteins are themselves stable under the conditions employed. In the case of vaccines, such as HBsAg bound to alumina, histidine and lactate were found to be excellent stabilizing additives, particularly in combination with phosphate.

The invention can be combined with other well established approaches to protein stability. For example, a protease inhibitor can be incorporated in the formulation to ensure that the protein is not slowly digested by protease activity present in the sample.

Another additive that may be used is a polyalcohol, e.g. at a concentration of at least 0.5%, and typically up to 5% (w/w). Examples of such compounds are saccharides such as inositol, lactitol, mannitol, xylitol and trehalose.

The ionic strength of the protein formulation stabilised by application of the present invention can be adjusted to meet the requirement for the intended use of the formulation (e.g. isotonic formulation for therapeutic use). Importantly, experiments showed repeatedly that in principle the stability of proteins at room temperature mirrors that at higher temperature, the rate of activity decline being many orders of magnitude 15 slower at room temperature compared with that at increased temperature (e.g. 60° C.).

### Adjuvant-Adsorbed Antigenic Proteins

All currently marketed formulations of hepatitis B vaccine are intended for storage at 2° C. to 8° C. These are typically liquid suspensions comprised of an aluminum hydroxide gel adjuvant and approximately 18 mM phosphate buffer adjusted to pH 7.0±0.1. Consequently, there is a certain degree of surface modification of the adjuvant in these formulations due to ligand exchange between surface hydroxides and the phosphate anion from the buffer. Such surface modification results in changes in pH microenvironment at the surface of the adjuvant particle and is therefore very likely to affect the stability of the antigen adsorbed on the adjuvant surface. The degree of the surface modification of adjuvant particles by phosphate has been reported to be proportional to the phosphate concentration in the formulation. The pH optimum for stability of HBsAg dissolved in aqueous solution was found to be around 8. However, if presented with aluminum hydroxide adjuvant in the absence of phosphate anions the stability of the antigen was very poor across the pH range between 5 and 9, showing only a marginally better stability at the acidic end of this pH range. The destabilizing effect of aluminum hydroxide adjuvant may be due to the previously reported alkaline pH microenvironment at the adjuvant surface. The present inventors demonstrated in this study that the stability of the adsorbed antigen is strongly dependent on the presence and concentration of phosphate in the formulation up to about 40 mM. Since no marked differences in stability were observed at higher phosphate concentrations, it appears that the presence of 40 mM phosphate is optimal to ensure good stability of the vaccine. It was also established that the neutral pH of the currently marketed hepatitis B vaccine is not optimal with respect to the stability of the adsorbed HBsAg antigen in the presence of phosphate. A slightly acidic pH resulted in markedly improved vaccine stability. Thus, the stability of the HBsAg adsorbed onto the aluminum hydroxide adjuvant can be considerably improved by formulating in the presence of 40 mM phosphate at a pH around 5.2. No measurements of the surface charge of the aluminum hydroxide adjuvant were carried out in this study, but it is believed that the main function of phosphate is in modification of the surface charge by ligand exchange, resulting in a pH microenvironment at the particle surface optimal for stability of adsorbed HBsAg in a suspension maintained at pH around 5.2.

In vitro activity of the adsorbed HBsAg did not appear to be adversely affected by the presence of 40 mM phosphate anion at pH 5.2 and was, surprisingly, comparable to that in the currently marketed vaccine preparation. Importantly, incubation at elevated temperatures did not result in changes of adsorbed portion of HBsAg which is another crucial aspect of stability of the vaccine in the new formulations based on 40 mM phosphate (pH 5.2). It was reported previously that in a phosphate-free formulation the adsorption of HBsAg onto aluminum hydroxide adjuvant is facilitated mainly by ligand exchange interactions between phospholipids of HBsAg and surface hydroxyls of the adjuvant. Electrostatic interactions were reported to play an insignificant role. It was important to establish in this study whether the surface modification of the adjuvant in the presence of phosphate anion affected the nature of the binding interactions between the antigen and the adjuvant. A potential increase in the importance of electrostatic binding at the expense of the ligand interactions could affect the binding efficiency depending on pH and ionic strength of the formulation with considerable implications for the quality of the vaccine. Electrostatic adsorption of protein antigens onto aluminum salt adjuvants has been described previously to be compromised by high ionic strength of the formulation. However, the present study demonstrated that the surface modification of the adjuvant by phosphate anion (40 mM) did not result in a marked increase of the importance of the electrostatic binding interactions as no considerable effect on the adsorbed portion of the antigen was observed in the presence of sodium chloride up to a 500 mM concentration. The adsorption thus appears to be facilitated by ligand interactions even in the presence of 40 mM phosphate anion, in the same way as reported previously for phosphate-free formulation. This means that ionic species, such as sodium chloride, can be used in the phosphate-based formulations to adjust osmolarity to an optimal level for therapeutic application.

The ionization state of phosphate depends on pH and is determined by its three dissociation constants: pKₐ₁=2.2, pKₐ₂=7.2, and pKₐ₃=12.3 at 25° C. (13). At a pH around 5.2, phosphate exists predominantly in the H₂PO₄⁻ ionic form and exerts a relatively small buffering capacity. In addition, due to its protonation state at pH 5.2 the buffering capacity of phosphate is stronger on the alkaline side than on the acidic side at such a pH level. Consequently, a formulation of HBsAg based on 40 mM phosphate at pH 5.2 requires additional buffering species. The ability of a chemical species to act as a pH buffer is defined by its dissociation constant (pKₐ) which should be within 1 pH unit from the target pH. However, even species whose pKₐ is between 1 and 2 pH units from the target pH can provide some degree of buffering capacity, especially if used at higher concentrations.

The stability of HBsAg adsorbed on aluminum hydroxide adjuvant in the presence of phosphate anion was shown to be further improved in the pH range between 4.6 and 5.2 by the presence of additional ionic excipients. While two of the additional excipients tested, namely malate anion having pKₐ₁=3.4 and pKₐ₂=5.1 at 25° C. and succinate anion having pKₐ₁=4.2 and pKₐ₂=5.5 at 25° C., possessed a pKₐ in the middle of the pH range studied and therefore provided a strong buffering capacity across the entire pH range, two other excipients, namely histidine having pKₐ₁=1.7, pKₐ₂=6.2 and pKₐ₃=9.1 at 25° C. and lactate ion having pKₐ=3.7 at 25° C., possessed a pKₐ outside the pH range. Consequently, these excipients provided optimal buffering capacity only in part of the pH range studied. However, in all cases, the buffering capacity of the formulation was considerably improved in the presence of the additional excipients over that present in the phosphate-only formulations.

It is noteworthy that the stability of HBsAg appeared to be particularly improved in the presence of excipients that did not comprise an ionizable group with pKₐ close (i.e., less than 1 unit) to the pH of the formulation (i.e., in the presence of lactate anion across the whole pH range studied and in the presence of histidine at pH 4.6 and 5.2). The presence of conventional buffers (i.e., species with pKₐ within one unit from the pH of the formulation), namely malate and succinate anions, had only a small or no effect on the stability over that observed in phosphate-only formulations. The apparent importance of the pKₐ in the stabilizing effect of the selected excipients suggests that exchange of hydrogen cations (H⁺) may have a role to play in the degradation of the HBsAg, because excipients with different pKₐ have a different ability of exchanging hydrogen cations with ionizable amino acids at the surface of HBsAg. It is believed that the improved stability achieved in the absence of components with pKₐ within 1 unit of the pH of the composition, i.e. in the absence of the conventional buffer, was due to the reduced rate of proton exchange at the surface of the antigen in such compositions compared with those in the presence of the conventional buffers.

The optimal formulation for hepatitis B vaccine developed in the course of this study to ensure stability of the antigen during storage comprises phosphate (40 mM) and either histidine (40 mM) or lactate (40 mM) and is adjusted to a pH about 5.2. The HBsAg stability in this formulation is considerably improved over that of the currently marketed hepatitis B vaccine compositions both under heat-stress at 55° C. and at elevated temperatures, including 37° C., 45° C. Importantly, all components of the stabilized formulations have a history of safe use in approved drug products, so they may not represent a hurdle in the regulatory approval of the stabilized formulation. Strong adsorption of antigen onto adjuvant is one of the key characteristics of a stable vaccine. It is therefore essential to ensure that the adsorption is not compromised by the inactive ingredients in the vaccine formulation. Importantly, it was shown that no significant antigenic activity was measured in the supernatants of the stabilized formulations, indicating that the HBsAg remains strongly bound to the adjuvant in the presence of these excipients. This is very likely due to the strong ligand exchange interactions between the phosphate groups of HBsAg and hydroxyl groups of the aluminum hydroxide adjuvant, even in the presence of 40 mM phosphate. Maintaining the appropriate size distribution of adjuvant particles is another key characteristic essential for the proper functioning of the vaccine, and is therefore one of the main quality parameters of any aluminum salt adjuvant-based vaccine. Importantly, no effect on the size distribution of vaccine particles was observed as a result of vaccine reformulation. The optimized formulation of hepatitis B vaccine reported here thus ensures improved heat stability of the antigen without compromising other key characteristics of the vaccines.

Thus, a formulation of hepatitis B vaccine was developed with improved stability at elevated temperatures, as measured by a validated in vitro potency test. The optimal formulation is adjusted to a pH around 5.2 and contains at least about 40 mM phosphate and at least about 40 mM histidine. Both phosphate anion and histidine have a history of safe use in approved drug products. While phosphate may be an important component in the formulation, the presence of histidine further improves the stability over that achieved in phosphate-only formulations. Lactate ion can be used in place of histidine to improve the stability of HBsAg. The use of histidine provides sufficient buffering capacity at pH 5.2 although due to the nearest pKa value (6.2) the buffering capacity of histidine at pH 5.2 is at its limit. Given the relatively high concentration of histidine, such buffering capacity is still sufficient to maintain the required pH. Interestingly, however, it was found that two buffers with optimal buffering capacity at a pH around 5.2 did not result in appreciable improvement of HBsAg stability. It is believed that the main function of phosphate is in modification of the surface of aluminum hydroxide adjuvant particle by ligand exchange resulting in a pH microenvironment at the particle surface optimal for stability of adsorbed HBsAg. The new formulation of hepatitis B vaccine was shown to be stable both at 37° C. and at 45° C. for at least 6 months using the in vitro AUSZYME potency test with acceptance criteria for testing hepatitis b vaccine potency validated against the in vivo test. The increased stability is very important not only in places where the cold chain is non-existent or insufficient, but also in places where the cold chain is established but temperature deviations may occur during transport or due to other interruptions.

It is believed that the results observed with HBsAg can be extrapolated to other formulations, particularly proteins, or antigenic proteins absorbed onto adjuvants, such as alumina. Thus, the invention relates to vaccine formulations, such as aqueous solutions, containing a protein absorbed onto an adjuvant (such as alumina) characterized by a retention of at least 50% of its activity when stored at a temperature of at least 45° C. for at least nine weeks. These formulations preferably contain a stabilizing effective amount of phosphate.

The following Example illustrates the invention.

### Example

### Hepatitis B Recombinant Vaccine

### Materials and Methods

### Vaccine

Hepatitis B vaccine (Shanvac-B) purchased from Shantha Biotech (Hyderabad, India) was used for this study. Each ml of the Shanvac-B vaccine contains about 20 μg of HBsAg, 0.5 mg of aluminum hydroxide adjuvant, 1.75 mg of disodium hydrogen phosphate, 0.88 mg of potassium dihydrogen phosphate, 7.71 mg of sodium chloride, and 25 μg of thimerosal. The vaccine has a pH between 6.9 and 7.1. Solution of HBsAg was obtained from Shantha Biotech.

### Materials

Deionized water (analytical reagent grade, Fisher, Loughborough, UK) was used in all experiments. L-histidine, sodium phosphate monobasic, dihydrate, sodium phosphate dibasic, dihydrate, and sodium lactate were from Fluka (Gillingham, UK). Sodium chloride, hydrochloric acid, and sodium hydroxide were from Fisher. Sodium malate, sodium succinate and TRIS were from Sigma (Poole, UK).

### Formulation and Stability Testing

New vaccine formulations were prepared by centrifuging (14,000 g, 5 min) the original vaccine formulation followed by washing the sediment once with water and resuspending in a background solution containing the new excipients. All background solutions were filtered through a 0.22 μm filter (Millipore, Bedford, Mass., USA) prior to their use in new vaccine formulations. The concentration of HBsAg and adjuvant in the new formulations were the same as those in the original Shanvac-B sample (i.e., 20 μg/ml and 0.5 mg/ml respectively). New vaccine formulations were finally transferred into 2 ml glass vials (2-CV, Chromacol, Welwyn Garden City, UK) and sealed with crimp caps. The sealed formulations were placed in an incubator adjusted to one of the following temperatures: 55° C. (Incufridge, Revolutionary Science, Lindstorm, Minn., USA), 45° C. (Lab-Line Instruments, Inc. Model No. 120, Melrose Park, Ill., USA), or 37° C. (Form a Scientific, Model No. 3326, Marietta, Ohio, USA) for allotted storage periods. Following the storage, the formulations were tested for remaining antigenic activity and in a particle-sizing assay.

The in vitro antigenic activity of the Hepatitis B vaccine was measured using the AUSZYME monoclonal diagnostic kit (Abbott Laboratories; cat no. 1980-64). The antigenic activity was determined both in the whole vaccine and in the supernatant following centrifugation (14,000 RPM, 5 min). Each sample was measured in triplicate. The antigenic activity was expressed as a percentage with respect to the value measured of the untreated refrigerated vaccine as follows:

$$\begin{matrix}R = \frac{( S - N )}{( C - N )} \times 100 \\ S = \frac{S 1 + S 2 + S 3}{3} \\ C = \frac{C 1 + C 2 + C 3}{3}\end{matrix}$$

wHere:
R is the recovery of antigenic activity (%)
N is the value of the negative control AUSZYME measurement
C1, C2 and C3 are the values of three repeated AUSZYME measurements of
the Control sample (i.e. untreated refrigerated vaccine)
S1, S2 and S3 are the values of three repeated AUSZYME measurements of the tested sample.

The initial formulation development was carried out by subjecting the vaccine to a heat-stress at 55° C. for 9 weeks to allow rapid identification of stability trends. The stability of neat HBsAg was shown to be optimal at pH around 8 in a histidine/TRIS buffer mixture. However, no such trend was observed when studying the effect of pH on adsorbed HBsAg. The stability of the adsorbed antigen was very low across the whole pH range between 5 and 9, showing a marginally better recovery following the heat-stress at the lower end of the pH range. This indicated a destabilizing effect of aluminum hydroxide adjuvant on HBsAg. The subsequent effort focused on investigation of the effect of different concentrations of phosphate anion on the stability of HBsAg adsorbed on aluminum hydroxide adjuvant. The effect was investigated across a pH range between 4.6 and 7.0, the lower end of this range being deemed an approximate limit of pH acceptability for a vaccine used in humans. Apart from phosphate, the formulations did not contain any other excipients, but the control formulations that did not contain phosphate anion comprised histidine (20 mM) as a buffer. The stability of adsorbed HBsAg was shown to be strongly dependent on the concentration of phosphate in the formulation (FIG. 1). Less than 20 percent of the original antigenic activity could be recovered after 2 weeks and less than 8 percent after 9 weeks of incubation at 55° C. in the absence of phosphate across the pH range studied. In contrast, more than 50 percent of the original antigenic activity was recovered after incubation of the vaccine in 20 mM phosphate at 55° C. for 2 weeks and more than 20 percent after incubation for 9 weeks. The stability was further improved in the presence of higher concentrations of phosphate leading to recovery of antigenic activity of greater than 70 percent after 2 weeks and greater than 40 percent after 9 weeks at 55° C. in the presence of either 40 mM or 100 mM phosphate. The stability appeared to be best in the pH range of 4.6 to 5.8, and no marked difference in the stability of HBsAg was observed between formulations containing either 40 mM or 100 mM phosphate at pH between 4.6 and 5.8. The stability at pH 6.4 and especially at pH 7.0 was considerably lower regardless of phosphate concentration.

The initial investigation of the effect of phosphate anion thus indicated two formulation parameters which are important for ensuring the good to excellent stability of HBsAg adsorbed on aluminum hydroxide adjuvant: (a) presence of at least 40 mM phosphate anion and (b) pH around 5.2. The adsorption of HBsAg onto the adjuvant did not appear to be adversely affected by the presence of 40 mM phosphate anion at pH 5.2 (Table I).

TABLE I
Effect of phosphate anion and sodium chloride on the
adsorption of HBsAg onto aluminum hydroxide antigen.

| | | Following 9 weeks |
|---|---|---|
| | Fresh sample | at 55° C. |
| Formulation | (% ± st. dev.) | (% ± st. dev.) |
| Original Shanvac-B (pH 7.0) | 97.1 ± 1.0 | 96.9 ± 0.6 |
| 40 mM phosphate (pH 5.2) | 96.8 ± 0.3 | 97.9 ± 0.7 |
| 40 mM phosphate (pH 5.2) + | 98.1 ± 0.4 | 96.8 ± 1.1 |
| NaCl (150 mM) | | |
| 40 mM phosphate (pH 5.2) + | 95.1 ± 0.9 | 93.9 ± 1.7 |
| NaCl (500 mM) | | |

The % adsorption is expressed as (T − S) × 100/T where T is the HBsAg activity in the whole vaccine and S is the HBsAg activity measured in the supernatant.

Furthermore, the presence of 150 mM NaCl in the 40 mM phosphate formulation had no effect of the adsorbed antigen, and only a limited effect was observed when the NaCl concentration was increased to 500 mM. The adsorption characteristics were not changed in any of these formulations following incubation at 55° C. for 9 weeks.

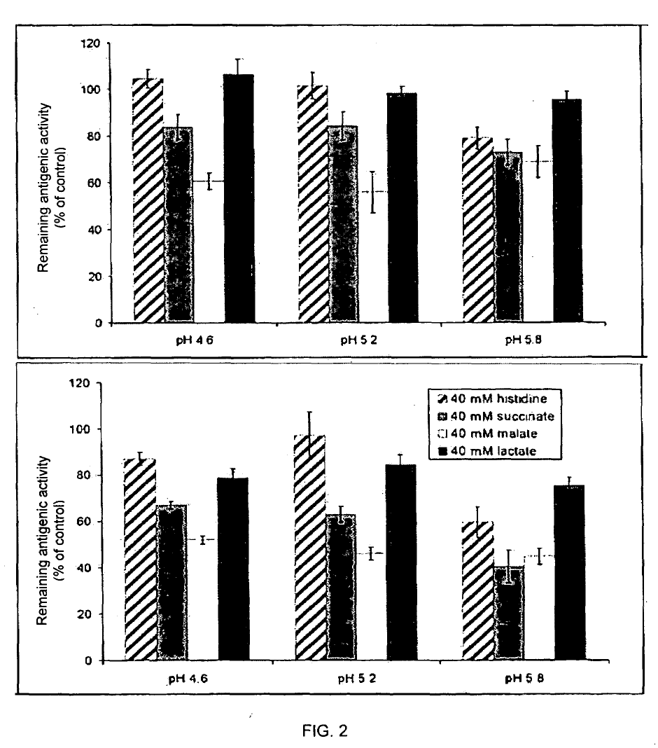

The effect of additional ionic excipients on the stability of HBsAg was investigated in the presence of 40 mM phosphate at pH 4.6, 5.2, and 5.8 covering the optimal conditions discovered during the initial formulation development. Ionic excipients were selected to improve the buffering capacity of the formulation as the buffering capacity of phosphate anion is very limited at a pH around 5.2. The presence of malate anion (40 mM) did not improve the stability of HBsAg at 55° C. over that achieved in the presence of phosphate anion only. The presence of succinate anion had a small beneficial effect on the HBsAg stability resulting in greater than 60 percent recovery of the antigenic activity following incubation at 55° C. for 9 weeks at pH 4.6 and 5.2. In contrast, the presence of lactate anion and particularly the presence of histidine resulted in considerably improved stability of HBsAg (FIG. 2). More than 85 percent of the antigenic activity was recovered following incubation at 55° C. for 9 weeks in the presence of lactate and more than 90 percent in the presence of histidine at pH 5.2. This pH appeared to be optimal, especially if histidine was used as the additional excipient. The stability was poorer at pH 5.8, but slightly better in the presence of lactate than in the presence of histidine.

No significant antigenic activity was measured in the supernatants of the formulations based on phosphate (40 mM) and either histidine (40 mM) or lactate (40 mM) at pH 5.2, indicating that the HBsAg remains bound to the adjuvant in the presence of these excipients (Table II).

TABLE II
Effect of histidine and lactate anion on the adsorption of HBsAg
onto aluminum hydroxide antigen in the presence of phosphate.

| | | Following 9 weeks |
|---|---|---|
| | Fresh sample | at 55° C. |
| Formulation | (% ± st. dev.) | (% ± st. dev.) |
| Original Shanvac-B (pH 7.0) | 96.8 ± 0.8 | 95.1 ± 1.9 |
| 40 mM phosphate + 40 mM | 97.4 ± 0.8 | 97.2 ± 1.0 |
| histidine (pH 5.2) | | |
| 40 mM phosphate + 40 mM | 95.6 ± 1.5 | 96.9 ± 1.1 |
| lactate (pH 5.2)+ | | |

The % adsorption is expressed as (T − S) × 100/T where T is the HBsAg activity in the whole vaccine and S is the HBsAg activity measured in the supernatant.

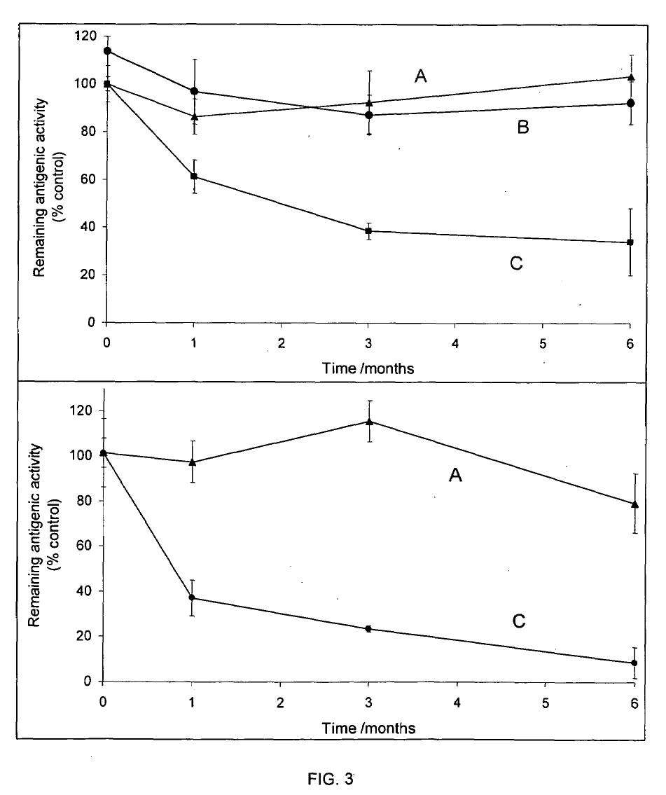

Lead candidate formulations which showed best stability under the heat-stress at 55° C., comprising histidine (40 mM) and phosphate (40 mM) at pH 5.2, were subsequently evaluated in stability studies at 45° C. and 37° C. The in vitro stability of the reformulated vaccine was compared with that in the original sample of Shanvac-B. The antigenic activity of the Shanvac-B vaccine dropped to approximately 60 percent after 1 month at 37° C. and about 30 percent after 6 months at 37° C. Similarly, the activity following incubation at 45° C. dropped to about 40 percent after 1 month and less than 10 percent after 6 months (FIG. 3). In contrast, the antigenic activity in the formulations based on histidine and phosphate remained at greater than 80 percent of the original level after incubation at 45° C. for 6 months and virtually at 100 percent after incubation for 6 months at 37° C. The stabilizing effect was not compromised by the presence of 100 mM sodium chloride at 37° C.

The in vivo immunogenicity of hepatitis B vaccine is dependent on both the stability of HBsAg and the particulate structure of the aluminum hydroxide adjuvant. It has been suggested that agglomeration of adjuvant particles affects the immunogenicity of vaccines and the size distribution of the adjuvant particles is therefore an important indicator of the aluminum salt adjuvant activity. Pairing the Coulter Counter results with in vitro and in vivo activities of the hepatitis B vaccine following various freeze-thaw treatments, we also observed this correlation. In the current study, the measured particle size distribution in the original vaccine formulation was as expected, >99% of the particles within the 1.5 to 3 micron range. Importantly, the change of the vaccine formulation did not appear to have an effect on the size distribution of vaccine particles (Table III).

TABLE III
Size distribution of aluminum hydroxide adjuvant particles with
adsorbed HBsAg antigen in the Shanvac-B vaccine and in the reformulated
vaccine containing histidine and phosphate.

| | Percentage of particles in each size range (μm) | | | |
|---|---|---|---|---|
| Sample | 1.5-3.0 μm | 3.0-6.0 μm | 6.0-9.0 μm | >9.0 μm |
| Original Shanvac-B | 99.80% | 0.19% | 0.01% | 0.00% |
| (pH 7.0) | | | | |
| Phosphate | 99.87% | 0.12% | 0.01% | 0.00% |
| (40 mM) + | | | | |
| histidine (40 mM), | | | | |
| pH 5.2 | | | | |

The distribution of the particle size was comparable between the original Shanvac-B formulation and the stabilized formulation based on histidine and phosphate at pH 5.2.

While this invention has been particularly shown and described with references to preferred embodiments thereof, it will be understood by those skilled in the art that various changes in form and details may be made therein without departing from the scope of the invention encompassed by the appended claims.

What is claimed is:

1. An aqueous vaccine composition comprising a protein adsorbed on a solid and one or more stabilising agents, further characterized in that
(i) the system is optionally substantially free of a buffer with pKₐ within 1 unit of the pH of the composition at the intended temperature range of storage of the composition;
(ii) the pH of the composition is set to a value at which the composition has maximum measurable stability with respect to pH; and
(iii) the one or more additives are capable of exchanging protons with the said protein and have pKₐ values at least 1 unit more or less than the pH of the composition at the intended temperature range of storage of the composition.

2. A composition according to claim 1, wherein the solid is an adjuvant.

3. A composition according to claim 1, wherein the solid is alumina.

4. A composition according to claim 3, comprising phosphate ions.

5. A composition according to claim 4, wherein the phosphate is at a concentration of at least 20 mM.

6. A composition according to claim 5, wherein the phosphate is at a concentration of at least 40 mM.

7. A composition according to claim 5, further comprising stabilizing agents having first and second groups with pKa values respectively higher and lower than the pH of the composition and are at least 50% of these groups are ionised.

8. A composition according to claim 7, wherein at least 80% of the groups are ionised, and preferably the groups are substantially completely ionised.

9. A composition according to claim 8, wherein the respective pKa values are each within 0.5 to 4 pH units of the pH of the composition.

10. A composition according to claim 9, wherein the respective pKa values are each within 1 to 3 pH units of the pH of the composition.

11. A composition according to claim 10, whose pH is between 4 to 9.

12. A composition according to claim 11 whose pH is about 5.2.

13. A composition according to claim 10, which comprises at least 0.1% (w/w) of each stabilising agent.

14. A composition according to claim 10, which comprises at least 0.5% (w/w) of each stabilising agent.

15. A composition according to claim 10, which comprises up to 200 mM of each stabilising agent.

16. A composition according to claim 10, which comprises up to 100 mM of each stabilising agent.

17. A composition according to claim 10, wherein the protein is an antigen.

18. A composition according to claim 17, wherein the antigen is an Hepatitis B antigen.

19. A composition according to claim 1, wherein the protein retains at least 50% of its immunogenicity after nine weeks of storage at 45° C. or more.

20. A composition according to claim 19, further comprising an aqueous solution, suspension or dispersion.

21. A composition according to claim 19, wherein the composition is a substantially dry formulation.

22. A composition according to claim 10, further comprising a polyalcohol.

23. A composition according to claim 22, which comprises at least 0.5% (w/w) of the polyalcohol.

24. A method of stabilizing a protein adsorbed onto a solid support comprising adding a stabilizing component which is attracted to the solid support surface and modifies the pH microenvironment to achieve a stabilized local pH.

25. The method of claim 24, wherein the solid support is alumina.

26. The method of claim 25, wherein the stabilizing component comprises phosphate ions.

27. The method of claim 26, wherein the phosphate is added at an amount of at least about 20 mM.
